(19) United States
(12) Patent Application Publication
(10) Pub. No.: US 2010/0152152 A1
(43) Pub. Date: Jun. 17, 2010

(54) Therapeutic Use of Nefopam

(76) Inventors: Michael Harvey Lyne, Essex (GB); Robin Mark Bannister, Essex (GB)

Correspondence Address: SALIWANCHIK LLOYD & SALIWANCHIK;A PROFESSIONAL ASSOCIATION, PO Box 142950, GAINESVILLE, FL, 32614, US

(21) Appl. No.: 11/996,790

(22) PCT Filed: Jul. 27, 2006
(86) PCT No.: PCT/GB2006/002828

§ 371 Date: Jan. 27, 2009

(30) Foreign Application Priority Data
Jul. 29, 2005 (GB) 0515703.7

(51) Int. Cl. A61K 31/33; A61P 25/24; A61P 25/22

(52) U.S. Cl. 514/183

(57) ABSTRACT

Nefopam is used for the manufacture of a medicament for the treatment of an affective disorder such as ADD or ADHD.

### FIELD OF THE INVENTION

This invention relates to a new therapeutic use of nefopam.

### BACKGROUND OF THE INVENTION

Nefopam i.e. 5-methyl-1-phenyl-3,4,5,6-tetrahydro-1H-2,5-benzoxazocine hydrochloride, is a centrally acting non-narcotic analgesic not structurally related to other analgesics. Nefopam has been shown to induce antinociception in animal models of pain and in humans.

In vitro and in vivo studies with nefopam enantiomers have shown that )+)-nefopam has more potent analgesic and dopamine, norepinephrine and serotonin uptake inhibitory properties than (−)-nefopam, with the order of potency given as (+)-nefopam>(±)-nefopam>(−)-nefopam (Fasmer et al., 1987; Rosland and Hole, 1990; Mather at al., 2001). In contrast to the study of Mather et al. (2001) who conclude that there is no compelling rationale to justify administering or monitoring individual enantiomers of nefopam, significant advantages of using the single enantiomers of nefopam have been shown for the treatment of pain and emesis. These utilities are disclosed in, inter alia, WO03/105832 and WO03/105833.

Conventional release preparations of nefopam have been commercially available for many years for use in moderate to severe pain, yet the short elimination half-life of nefopam (four hours) means that it is difficult to maintain analgesic efficacy over the normal dosing period (three times daily). Dose escalation of nefopam brings about an increase in the frequency of adverse drug reactions, and adverse effects on pulse and blood pressure have been observed following parenteral delivery of therapeutic doses of nefopam (Heel et al., 1980). The chronotropic and ionotropic effects of nefopam on the heart are not present when nefopam is administered orally (Bhatt et al., 1981).

Attention-deficit disorder (ADD) and attention-deficit hyperactivity disorder (ADHD) are common conditions, especially among children. Methylphenidate is used in treatment but may cause side-effects. Related conditions include Tourette's disorder, juvenile behavioural disorders (such as oppositional defiant disorder, conduct disorder and persuasive child development disorder), anxiety disorders and eating disorders (such as anorexia, binge-eating disorder and bulimia).

PCT/GB2006/001197 (unpublished at the date of filing this Application) discloses the use of nefopam in the treatment of fibromyalgia.

### SUMMARY OF THE INVENTION

The present invention is based on the realisation that nefopam may have utility in the treatment of an affective disorder, e.g. ADD, ADHD, Tourette's disorder, juvenile behavioural disorders (such as oppositional defiant disorder, conduct disorder or persuasive child development disorder), anxiety disorders and eating disorders (such as anorexia, binge-eating disorder and bulimia). Controlled release may extend the effect and reduce the occurrence of side-effects associated with plasma peak concentrations of an immediate release product. \

### DESCRIPTION OF PREFERRED EMBODIMENTS

As used herein, “nefopam” refers to a compound of formula I

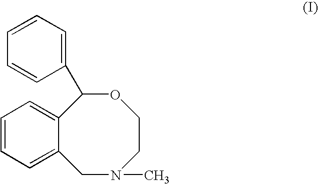

and salts, e.g. the hydrochloride, metabolites and prodrugs thereof, as well as the (+) and (−) enantiomers which are as far as possible optically pure. (+)-Nefopam may be preferred, e.g. for reduced side-effects that may be caused by interaction.

An analogue of nefopam may be used. Such compounds are described in WO2004/056788 and WO2005/103019.

According to the invention, the active compound is used in a method of treating an affective disorder. The term “affective disorder” includes the major affective disorders that are generally understood to cover bipolar disorder, unipolar disorder and schizoaffective disorders, and also affective spectrum disorders which are a grouping of related psychiatric and medical disorders which may accompany major affective disorders at statistically higher rates than would normally be expected. These disorders are identified by a common positive response to the same types of pharmacologic treatments and aggregate strongly in families, and may therefore share common heritable underlying physiologic anomalies.

Accordingly, relevant medical conditions include major affective disorders such as bipolar disorder (manic depression), unipolar disorder (depression), and schizophrenia; affective spectrum disorders such as attention-deficit hyperactivity disorder, body dysmorphic disorder, bulimia nervosa and other eating disorders, cataplexy, and dysthymia; and general anxiety disorders such as hypersexuality, impulse-control disorders, irritable bowel syndrome, kleptomania, multiple chemical sensitivity, narcolepsy, obsessive-compulsive disorder, panic disorder, post-traumatic stress disorder, premenstrual dysphoric disorder and social phobia.

The following may also be part of the spectrum accompanying affective disorders, i.e. autism, chronic pain, Gulf War syndrome, intermittent explosive disorder, pathological gambling, personality disorder, pyromania, substance abuse and addiction (including alcoholism), and trichotillomania.

In particular, nefopam is used according to the present invention in a method of treating ADD, ADHD, Tourette's disorder, juvenile behavioural disorders (such as oppositional defiant disorder, conduct disorder or persuasive child development disorder), anxiety disorders and eating disorders (such as anorexia, binge-eating disorder or bulimia). The patient may be any in need of treatment, e.g a hyperactive child.

Any suitable route of administration can be used. For example, any of oral, topical, ocular, rectal, vaginal, inhalation and intranasal delivery routes may be suitable. The dose of the active agent will depend on the nature and degree of the condition, the age and condition of the patient, and other factors known to those skilled in the art. A typical dosage is at least 1 mg, e.g. 10 to100 mg, given one to three times per day. A typical dosage for an immediate release oral formulation is at least 1 mg, e.g. 10 to 100 mg, given one to three times per day. For a modified release formulation, a typical dosage is at least 1 mg, e.g. 10 to 400 mg, given once or twice per day.

If controlled release of the active agent is required, a suitable formulation of any type known to those skilled in the art may be used. Modified release can be afforded by either dissolution or diffusion-controlled monolithic devices, beaded encapsulated systems, osmotically controlled systems, and modified film coating systems incorporating suitable polymeric and non-polymeric hydrophilic and hydrophobic materials. Suitable controlled-release formulations include hydrophilic materials comprising, but not limited to, acrylic or methacrylic polymers or copolymers, alkylvinyl polymers, celluloses, hydroxyalkyl celluloses, carboxyalkyl celluloses, polysaccharides, alginates, pectins, starches and derivatives, natural and synthetic gums, polycarbophil, chitosans. Suitable hydrophobic materials comprise, but are not limited to, hydrophobic polymers, waxes, fats, long-chained fatty acids, their corresponding esters, their corresponding ethers, and their mixtures.

It will often be advantageous to use nefopam in combination with another drug. Such another drug may be a psycho-stimulant (such as dextroamphetamine, amphetamine, methylphenidate, pemoline or dexmethylphenidate), a central alpha agonist (such as guanfacine, tolonidine, talipexole, tiamenidine, linamidine, clonidine or tizanidine) or a monoamine re-uptake inhibitor antidepressant (such as atomoxetine, imipramine, desipramine, reboxetine or bupropion).

The following Example illustrates the invention.

Nefopam and (+)-nefopam were evaluated in the Marble Burying Test, a model which detects anxiolytic/tranquillizing activity. This is a general model for affective disorders.

The method follows that described by Broekkamp et al. (1986 Eur. J. Pharmacol., 126, 223-229. Mice exposed to novel objects (marbles) will bury them in the sawdust floor covering. Anxiolytics decrease the number of marbles buried at non-sedative doses.

Mice are individually placed in transparent plastic cages (33×21×18 cm) with 5 cm of sawdust on the floor and 25 marbles grouped in the centre of each cage. Each test cage is covered with an inverted plastic covering. Each test cage, together with the marbles, is impregnated with mouse odor beforehand, by leaving 10 mice in the cage for 15 minutes. These mice then play no further role in the experiment. The number of marbles covered by sawdust (⅔ or more) is counted at the end of a 30 minute test.

All compounds were administered i.p. 30 minutes before the test, and compared with a vehicle control group. Clobazam (8 mg/kg i.p.), administered under the same experimental conditions, was used as reference substance.

| Number of marbles buried | | | |
|---|---|---|---|
| (% change from ctrl) | Nefopam | (+)-nefopam | Clobazam |
| 10 mg/kg ip | −66* | −56* | Nt |
| 20 mg/kg ip | −98* | −99* | Nt |
| 32 mg/kg ip | nt | nt | −75* |
| 40 mg/kg ip | −100* | −100* | Nt |

nt = not tested;
*Denotes statistical significance achieved

These positive data indicate that both nefopam and (+)-nefopam may have utility in ADHD and related conditions.

1. Use of nefopam for the manufacture of a medicament for the treatment of an affective disorder.

2. Use according to claim 1, wherein the disorder is attention-deficit disorder or attention-deficit hyperactivity disorder.

3. Use according to claim 1, wherein the disorder is Tourette's disorder, a juvenile behavioural disorder, an anxiety disorder or an eating disorder.

4. Use according to any preceding claim, wherein the medicament provides controlled or delayed release of the nefopam.

5. Use according to any preceding claim, wherein the nefopam is the (+) enantiomer, substantially free of (−)-nefopam.
